(19) United States
(12) Patent Application Publication
(10) Pub. No.: US 2006/0292205 A1
(43) Pub. Date: Dec. 28, 2006

(54) Substitute for animal protein in cattle feed

(76) Inventor: Leanne Gail Robinson, Madrid, NY (US)

Correspondence Address: Donavon Lee Favre, Philip Benjamin Tower, 250 58th St N, Apt 1009, St Petersburg, FL, 33710, US

(21) Appl. No.: 10/530,630

(22) PCT Filed: Oct. 8, 2002
(86) PCT No.: PCT/US02/32078

§ 371 Date: Apr. 7, 2005

(51) Int. Cl. A23K 1/165; A61K 38/43

(52) U.S. Cl. 424/442; 424/93.45; 424/94.1

(57) ABSTRACT

A combination one or more species of lactobacillus bacteria and one or more types of fibrolytic enzymes can be used to replace animal protein in cattle feed. The combination results in a better amino acid balance in the digestive tract of cattle resulting in a better utilization of nitrogen. Less water-soluble nitrogen compounds pass through the digestive tract resulting in less pollution. More nitrogen in the feed is converted to water insoluble compounds resulting in better feed utilization and less water-soluble pollution in the manure. The diseases carried by some animal protein additives, such as “mad cow” are not present in the additives of the present invention.

### TECHNICAL FIELD

The field of the present invention is cattle feed additives, which improve feed nitrogen utilization, and eliminate the need for animal protein supplements, which animal protein supplements can cause disease.

### BACKGROUND ART

U.S. Pat. No. 5,780,288 Rohwer (1998) discloses at Col. 1, line 15 to Col. 2, line 20, “Creutztfeldt-Jakob Disease (CJD) is a rare neurological disease found in humans, first described in the 1920s and found worldwide. It is usually manifested in late middle-age with progressive dementia and is usually fatal within six months. It is characterized by spongiform changes in the brain, but this can only readily be diagnosed at post mortem. The identification in 1996 of at least 10 cases of CJD in Britain which seem to represent a new variant caused concern that these cases could be linked to exposure to bovine spongiform encephalopathy (BSE), or “Mad Cow Syndrome,” which has infected some 160,000 cows in Britain. The distinct variant in these 10 cases occurred in people aged under 42 with dates of onset of illness in the last two years. This variant has not been previously recognized and is characterized by behavioral change, ataxia, progressive cognitive impairment and a tendency to a prolonged duration of illness. In April 1996, Dr. Stanley B. Prusiner of the University of California, San Francisco, presented scientific evidence that he believes indicates a link between CJD and BSE.

In 1988, as a result of earlier concerns about the possible transmission of BSE to humans, the UK adopted control measures which included: 1) destroying cattle clinically diagnosed on the farm, 2) prohibiting feeding cattle and other ruminants material containing animal protein derived from ruminants and 3) destroying carcasses of cattle infected with BSE. The public health threat of the 10 new cases was deemed great enough that the European Union (EU) has imposed further precautionary measures which include: 1) a ban on the international sale of all meat, offal, semen, embryos, and other products of British cattle, 2) a requirement that carcasses from cattle aged over 30 months must be destroyed, and 3) a prohibition on the use of mammalian meat and bone meal in feed for all farm animals.

The health panic triggered by the evidence that the fatal CJD might be caused by eating beef has fast become a significant economic issue. The cost to the UK and EU alone of destroying cattle which are aged over 30 months is predicted to be approximately $10 billion if the cattle are killed at a rate of 15,000 per week over the next six years.

There have been no reported cases of BSE in the United States; nonetheless, as a preventative measure, a prohibition of imported ruminants from the U.K. was implemented in July 1989. Scrapie and other forms of spongiform encephalopathy are present in the United States, however, causing an intense interest in BSE. Presently, a voluntary practice against feeding ruminant byproducts to ruminants exists in the U.S. There is ongoing discussion among governmental regulatory agencies on whether to impose an official ban on such feeding practices. A related issue of concern in the U.S. is that a transmissible form of spongiform encephalopathy found in ranched mink, Transmissible Mink Encephalopathy (TME), has been primarily attributed to feeding the mink-scrapie-infected sheep and goat carcasses, Cattle carcasses, which are also part of the ranched mink diet, are now a suspected source of TME (Bolis & Gibbs J. Amer. Vet. Med. Assoc., 1990).

BSE is believed to be caused by a biological agent called a prion protein. Prions are unique to the world of biology because they are able to replicate without the benefit of any nucleic acid (e.g., DNA or RNA). Nucleic acids are used by everything from viruses to bacteria to humans to store genetic information. This genetic information is used by organisms to build specific proteins. Proteins, in turn, do the work of the cell. Normally, proteins do not have the ability to vary genetic information. What makes prions very unusual is that they seem to be made exclusively of proteins. Since prions are able to propagate themselves, it is believed that the prion proteins are able to carry genetic information. The unusual ability of prions to possibly carry genetic information in their proteins probably explains the unique etiology of BSE and other diseases caused by prions.”

In the book, The Secret Life of Germs, by Philip M. Tierno Jr., Ph.D., Simon & Shuster, (2001) page 139 it is disclosed, “With regard to human prion disease, or CJD, the thought of prions eating holes in your brain after thirty or forty years is frightening enough, surely. But in 1996 (CJD) began to kill British people in their late teens and early twenties. When these cases of variant CJD (vCJD) were lie to eating British beef the mad cow scare began in earnest. “You eat it. Then it eats you” was the message the media shrieked into the public's ear. Meanwhile the British authorities reacted slowly, delaying the implementation of a necessary temporary ban on the sale of British beef. Even after the ban went into effect, including a ban on the domestic sale of feed containing animal parts. The British government allowed that same feed to be exported to other countries, including the United States.”

On page 140, Tierno states, “—there have been several reports in recent years that elk, deer, and moose in the Western United States and Canada are dying of chronic wasting disease (CWD), which is apparently identical with prion disease in cattle. It has been speculated that the disease got into these animal populations because of contaminated British feed sold to North American game farms.”

The following articles dated 2002 from the New York times document the spread of “Mad Cow” disease. “ENVER, August 6—Wildlife experts from the United States and Canada are meeting here to discuss strategies for containing the spread of chronic wasting disease, the variant of mad cow disease that kills deer and elk. The malady, once found only in the brushy foothills near Fort Collins, Colo., has now been identified in both captive and wild herds of deer and elk in Kansas, Montana, Nebraska, New Mexico, Oklahoma, South Dakota, Wisconsin and Wyoming and the Canadian provinces of Saskatchewan and Alberta” “WAUSAU, Wis., July 31—The deaths of three outdoorsmen from brain-destroying illnesses are under investigation by medical experts who want to know whether chronic wasting disease has crossed from animals into humans, just as mad cow disease did in Europe.”

U.S. Pat. No. 5,093,121 of Kvanta, et al. Mar. 3, 1992 entitled “Method for increasing the protein contents of milk in ruminants” discloses at the Abstract, “The invention relates to a method of increasing the protein contents of milk in milk producing animals by introducing into the animal a culture of one or more non-pathogenic lactic acid producing live bacteria in admixture with a carrier. The invention also relates to a preparation containing said lactic acid producing bacteria in admixture with a carrier facilitating the optimal growth of the bacteria in the stomach-intestine system of the animal. The invention also deals with the use of non-pathogenic lactic acid producing live bacteria for increasing the protein contents of milk in milk producing animals.

U.S. Pat. No. 5,529,793 of Garner, et al., Jun. 25, 1996, entitled, “Compositions for Improving the Utilization of Feedstuffs by Ruminants,” discloses at the abstract, “A composition of a mixture of a lactic acid producing bacteria culture and a lactate utilizing bacteria culture admixed with a dry formulation or an animal feedlot diet for improving the utilization of feedstuffs by a ruminant. The composition may be used on a continual basis to increase meat or milk production, or used during the transition from a roughage diet to a feedlot diet to prevent or minimize acidosis. The preferred embodiment utilizes Lactobacillus acidophilus as its lactic acid producing bacteria culture and Propionibacterium P-5 as its lactate utilizing bacteria culture. The composition is in a dry powder form for storage at ambient temperatures for long durations.”

U.S. Pat. No. 5,662,901 Tobey et al (1997) discloses at the Abstract, “The invention comprises two grain conditioners. The first grain conditioner, which is suitable for all grains, comprises a pectinase, a protease, a beta-glucanase and an amylase. The second grain conditioner, which is designed for use on easier-to-digest grains, comprises a pectinase, a beta-glucanase, an amylase and a hemicellulase.”. At col. 1, lines 58 to 60 Tobey et al “WAUSAU, Wis., July 31—The deaths of three outdoorsmen from brain destroying illnesses are under investigation by medical experts who want to know whether chronic wasting disease has crossed from animals into humans, just as mad cow disease did in Europe.”

U.S. Pat. No. 5,093,121 of Kvanta, et al. Mar. 3, 1992 entitled “Method for increasing the protein contents of milk in ruminants” discloses at the Abstract, “The invention relates to a method of increasing the protein contents of milk in milk producing animals by introducing into the animal a culture of one or more non-pathogenic lactic acid producing live bacteria in admixture with a carrier. The invention also relates to a preparation containing said lactic acid producing bacteria in admixture with a carrier facilitating the optimal growth of the bacteria in the stomach-intestine system of the animal. The invention also deals with the use of non-pathogenic lactic acid producing live bacteria for increasing the protein contents of milk in milk producing animals.

U.S. Pat. No. 5,529,793 of Garner, et al., Jun. 25, 1996, entitled, “Compositions for Improving the Utilization of Feedstuffs by Ruminants,” discloses at the abstract, “A composition of a mixture of a lactic acid producing bacteria culture and a lactate utilizing bacteria culture admixed with a dry formulation or an animal feedlot diet for improving the utilization of feedstuffs by a ruminant. The composition may be used on a continual basis to increase meat, or milk production or used during the transition from a roughage diet to a feedlot diet to prevent or minimize acidosis. The preferred embodiment utilizes Lactobacillus acidophilus as its lactic acid producing bacteria culture and Propionibacterium P-5 as its lactate utilizing bacteria culture. The composition is in a dry powder form for storage at ambient temperatures for long durations.”

U.S. Pat. No. 5,662,901 Tobey et al (1997) discloses at the Abstract, “The invention comprises two grain conditioners. The first grain conditioner, which is suitable for all grains, comprises a pectinase, a protease, a beta-glucanase and an amylase. The second grain conditioner, which is designed for use on easier-to-digest grains, comprises a pectinase, a beta-glucanase, an amylase and a hemicellulase.” At col. 1, lines 58 to 60 Tobey et al disclose, “The addition of enzymes to animal feeds is also known to increase feed utilization efficiency and weight gain.”

U.S. Pat. No. 5,720,971, Enzyme Additive for Ruminant Feeds, Beauchemin et al (1998) discloses at the abstract “—The fibrolytic enzyme supplements consist of mixtures of cellulase and xylanase is certain preferred ratios—. The cellulase and xylanase are dissolved in a buffer and sprayed onto dry legume forages or grain feeds. The feed material is then incubated for at least three hours to allow the enzymes to be absorbed into and adhere to the feed material.”

Beauchemin also discloses, “Direct addition of fibrolytic enzymes to the ruminal environment is also unlikely to be of benefit as the rumen contains bacteria, fungi and protozoa which produce the most active cellulase and xylanase known to exist in any environment (Gilbert 1992).”

In an article entitled Fibrolytic enzymes for beef and dairy cows, David Hutcheson PhD., Animal Agricultural Consulting, Inc. PO Box 50367 Amarillo, Tex. 79159 discloses, “Fibrolytic enzymes increases dry matter digestibility, neutral detergent fiber digestion, organic mater, cellulose, hemicellulose and increase ruminal rates of microbial protein.”

U.S. Pat. No. 6,221,381 B1 Shelford (2001) discloses at the Abstract “Methods and compositions are provided for enhancing feed utilization efficiency in a ruminant animal by adding to the feed a sufficient amount of a nonionic surfactant to enhance the utilization of the feed by the animal.—A digestion enhancing enzyme and lactic acid bacteria inoculum may also be added to the feed.” At col. 7, first paragraph, Shelford discloses “In addition to feed and a nonionic surfactant, the compositions of the invention may further comprise one or more additional agents that enhance the ruminant digestive processes. Such agents include, for example, pyrodoxal 5-phosphate, fumaric acid and its salts, sorbic acid and its salts, parabenzoic acid esters, benzoic acid, polydimethyl siloxane-polyethers, unsaturated alcohols, bentonite, proteolytic and/or carbohydrase enzymes, such as glycanase, hemicellase, cellulase, pectinase, xylanase and amylase, and lactic acid bacteria inoculants, such as those comprising Lactobacillus casei, L. acidophilus, L. salivarius, L. corymiformis subsp coryniformis, L. curvatus, L. plantarum, L. brevis, L. buchneri, L. fermentum, L. viridescens Pdiococcus acidilacti, P. cerevisiae, P. pentosaceus, Streptococcus faecalis, S. faecium, S. lactis, L. buchneri, L. fermentum, L. viridenscens, L. delbrueckiin, Leuconostoc cremoris, L. dextranicum, L. mesenteroides or L. citrovorum. Where the surfactant is used in conjunction with exogenous glycanases, the method of producing feed compositions in the sent invention is most effective when surfactant constitute on the order of about 0.01% of the dry weight of the feed. In situations where the surfactant is used without exogenous enzymes, the compositions are most effective when the surfactant concentration does not exceed about 0.2%, of the dry weight of the feed.”

U.S. Pat. No. 6,017,525 Logan et al (2000) discloses at the abstract, “A dry composition containing large numbers of beneficial bacteria and enzymes for the digestion of poultry manure is used to treat poultry litter. The growth of the beneficial bacteria, activated by moisture in the poultry droppings prevents the growth of pathogenic bacteria such as E. coli, Salmonella and Campylobacter by competitive exclusion.”

U.S. Pat. No. 5,876,990 Reddy et al (1999) discloses at the Abstract, “A first media provides an oxygen inducer such as catalase—. A second media provides an oxygen supplier such as a peroxide.” The composition can also contain ermines and bacteria. At col. 29, Example 12, Reddy discloses, “This experiment evaluated the effect on growth, production, general well being, and reduction of mortality of adding oxy-prep and micro-prep to feed for beef cattle, dairy cattle, poultry, dogs, cats, and pigs. The micro-prep was prepared according to the procedure described under Composition of Micro-Prep, above. The following micro-organisms were grown individually: Lactobacillus acidophilus, Lactobacillus casei, Lactobacillus plantarum, Pedicoccus acidolactic, Lactococcus lactis var. lactis, Bifidobacterium bifidus, Lactococcus lactis var Lactis subspecies diacetyllactis, streptococcus faecium, Propionibacterium shermanii, Propionibacterium arabinosum and Propionibacterium zeae, Saccharomyces cerevisiae, Aspergillus oryza and Bacillus subtilus. At the end of the growth, the organisms were mixed together, forming a combined liquid culture. Ten gallons of the combined liquid culture was thoroughly mixed with the following ingredients to form a doughy mass: 1.0 pounds of lecithin, 0.1 pounds of sodium propionate, 2.0 pounds calcium carbonate, 2.0 pounds of multi-enzymes, 0.10 pounds of yucca schidigera extract (range 0.01 to 1 pound), 40 pounds of sodium bentonite (range 30 to 60 pounds), 20 pounds of rice flour (range 10 to 30 pounds), and 20 pounds of wheat flour. The pH of the mix was adjusted to 6.5 to 7.5 using sodium hydroxide or sodium bisulfate. The micro-prep was extruded in the form of small pellets. The extruded micro-prep was dried and milled to the consistency of the feed.” At claim 6, the enzyme was disclosed as follows, “6. The media system of claim 5, wherein said pellet further comprises an enzyme selected from the group consisting of protease, lipase, amylase, cellulase, pectinase, glucose oxidase, galactose oxidase, lactase, and mixtures thereof.”

The present inventors market a product containing lactobacillus and an enzyme system. The composition was a trade secret. The ratio of digestive enzyme units to colony forming units is estimated to be 6.8 digestive enzyme units to 10⁷ colony forming units based upon the input ingredients. The amount of enzyme per feeding was 2.7×10³ digestive units per oz. (28.3 g).

In addition to mad cow disease, it is postulated that many other diseases are transmitted to cattle by feeding animal protein and other animal derived products such as poultry manure to the cattle. The statistics on food poisoning caused by infected meat in the United States are appalling, in spite of all the U.S. Government regulations.

On page 141 Tierno states, “According to the federal Government's General Accounting Office, the majority of companies in the U.S. cattle industry, perhaps seventy percent of the total, do not comply with the existing rules.”

The U.S. Government is attempting to control the spread of “wasting disease” (Mad Cow?) in the wild. One such system is the establishment of “elimination zones”.

Another serious problem, which is well known, is water pollution caused by cattle manure and urine. The source of much of the pollution is undigested, water-soluble nutrients that pass through the cattle. In the following claims, water soluble nitrogen compounds exclude nitrogen compounds in which the nitrogen is fixed in a polymer or in a peptide.

### DISCLOSURE OF THE INVENTION

The present invention is directed to cattle feed additives formulated to replace disease carrying animal protein additives that have been used in the past. The additive of the present invention eliminates the transmission of disease caused by animal protein additives, is equivalent to animal protein in the digestion of food by cattle and reduces the amount of water soluble undigested waste which passes through the cattle, thus reducing ground and water pollution. The primary advantage of the present invention is increasing ruminal microbial efficiency thus eliminating the need for dangerous bypass protein, which is very expensive, and improving the efficiency on protein utilization. Without being bound by theory, it is postulated that the enzymes of the present invention cause a rapid break down of material, including fiber, in the rumen to provide food for the bacteria, causing a rapid growth of bacteria which are high quality microbial protein. This was a totally unexpected result. As this protein is cheaper to produce than a comparable grade of animal protein is to buy, it creates an incentive to feed cattle the cattle feed additive of the present invention instead of diseased bypass protein.

The present invention is a cattle feed additive containing fibrolytic enzymes having enzyme activity and one or more species of lactobacillus bacteria having colony forming units wherein the ratio of enzyme activity to colony forming units has a value of at least about 1 unit of digestive enzyme activity to every 10⁵ colony forming units. Preferably the cattle fed additive has a ratio of enzyme activity to colony forming units has a value of at least 2 units of enzyme activity to every 10⁶ colony forming units. Preferably the lactobacillus bacteria are selected from the group comprising Lactobacillus Acidophilus, Lactobacillus Plantarum, and Lactobacillus Brevis, and mixtures thereof. Preferably the fibrolytic enzymes are selected from the group comprising cellulases, xylanase, hemi-cellulase and mixtures thereof.

The composition of the present invention can be free of surfactants and any other ingredients disclosed in the prior art to enhance the performance of enzymes and/or lactobacillus bacteria.

The method of making cattle feed of the present invention is characterized by replacing previously used bypass protein in the animal feed with a sufficient amount of a mixture of one or more specie of lactobacillus bacteria and one or more types of fibrolytic enzymes, to produce at least enough microbial protein to be at least equivalent to one half pound (0.23 kg) of animal protein fed to each of the cattle per day, assuming that each of the cattle are mature and of an average weight for cattle. The preferred lactobacillus bacteria are selected from the group consisting of Lactobacillus Acidophilus, Lactobacillus Plantarum, and Lactobacillus Brevis, and mixtures thereof, and the protein byproducts replaced are selected from the group consisting of nerve, brain, blood, bone and meat containing byproducts. The preferred lactobacillus bacteria are a mixture of Lactobacillus Acidophilus, Lactobacillus Plantarum, and Lactobacillus Brevis. The one or more digesting enzymes are preferably selected, from the group consisting of xylanase, and cellulases derived from Trichoderma viride, Aspergillus oryzae, Aspergillus niger, and Bacillus subtilis. Preferably the one or more digesting enzymes are a mixture of xylanase, and cellulases derived from Trichoderma viride, Aspergillus oryzae, Aspergillus Niger, and Bacillus subtilis.

The method of converting cattle feed to microbial protein in cattle of the present invention is also characterized by incorporating a sufficient amount of a mixture of one or more species of lactobacillus bacteria and one or more types of digesting enzymes into cattle feed to form at least a sufficient amount of microbial protein to be at least equivalent to one four pound (0.11 kg) of animal protein fed to each of the cattle per day. The lactobacillus bacteria are preferably selected from the group consisting of Lactobacillus Acidophilus, Lactobacillus Plantarum, and Lactobacillus Brevis, and mixtures thereof and the amount of microbial protein formed is at least equivalent to one half pound (0.23 kg) of animal protein fed to each of the cattle per day. The lactobacillus bacteria are preferably a mixture of Lactobacillus Acidophilus, Lactobacillus Plantarum, and Lactobacillus Brevis. The one or more digesting enzymes are preferably selected from the group consisting of xylanase, and cellulases derived from Trichoderma viride, Aspergillus oryzae, Aspergillus niger, and Bacillus subtilis. One or more digesting enzymes are preferably a mixture of xylanase, and cellulases derived from Trichoderma viride, Aspergillus oryzae, Aspergillus Niger, and Bacillus subtilis.

In another embodiment the cattle feed of the present invention is characterized by the daily ration of feed fed to each head of cattle containing a sufficient amount of one or more strains of lactobacillus bacteria and one or more types of digesting enzymes having an enzyme activity of at least 10⁴ digestive units per oz (28.35 g) to digest the cattle feed for the bacteria and increase the number of bacteria in the rumen which are microbial protein. Preferably the enzymes are present at a level sufficient to produce an enzyme activity of from 10⁴ to 10⁸ units per gram of cattle feed and the lactobacillus bacteria being present at a level sufficient to increase the yield of microbial protein in the rumen. The microbial protein is produced in the cattle by interaction of the bacteria and enzymes, the bacteria are preferably present at a level of from 10⁶ to 10¹⁰ colony forming units per gram of cattle feed and enzymes are preferably present at a level sufficient to produce a digestive enzyme activity of from 10⁶ to 10⁷ units per gram of cattle feed.

The method of the present invention reduces runoff of water soluble nitrogen compounds from cattle manure comprising by incorporating a sufficient amount of a mixture of one or more species of lactobacillus bacteria and one or more types of digesting enzymes into cattle feed to form at least a sufficient amount of microbial protein to be at least equivalent to one fourth pound (0.11 kg) of animal protein fed to each of the cattle per day. This provides a better amino acid balance and the production of insoluble nitrogen compounds. The cattle feed of the present invention can be, free of a surfactant on a carrier.

### MODES FOR CARRYING OUT THE INVENTION

The Protein Edge™ (PE) feed additive of the present invention contained the following ingredients. The active ingredients portion contained 8.8 pounds (4 kg) of Ruminant Formula 40 AF and 80 pounds (36.3 Kg) of M8C enzymes.

Ruminant Formula 40 AF contains a mixture of Lactobacillus Acidophilus, Lactobacillus Plantarum, and Lactobacillus Brevis. These are live, concentrated bacteria suspended in a mixed sugar base. The bacteria are in a weight % ratio of Lactobacillus Acidophilus 60%, Lactobacillus Plantarum 20%, and Lactobacillus Brevis 20%. The final concentration with the sugar base is blended to 80 billion cfu/gram with a guarantee of 40 billion cfu/gram. The bacteria were prepared according to the procedure of U.S. Pat. No. 4,226,940 Storrs (1980).

M8C enzymes are a dried fermentation extract of Bacillus subtilis, Aspergillus oryzae, Trichoderma viride and Aspergillus niger. M8C enzymes are a 50/50 mixture by weight of EX 28000 enzymes and Multicel 185 enzymes.

Enzymes that can be used in the practice of the present invention are available firm NOVOZYMES JEFFREYS BIOLOGICALS, Inc. Salem, Va. in products known as Xylanase, Maxicel™, Multicel™ and EX 28000™ enzymes.

Xylanase acts on D-Xylan in a manner reminiscent of alpha and beta amylase on starch and results in the production of D-Xylose. Due to interactions with pectin and hemicellulose, Xylanse has a considerable amount of pectinase added. The activity is 30,000 to 150,000 Xylanase Units/g. This product also contains high levels of cellulase, pentosanase and petinase.

Many cellulases of fungal orgin are known for their activity range extending well into the lower pH values. Maxicel and Multicel are two such companion products with very concentrated cellulolytic activity.

Multicel™ 185 cellulase is a combination of cellulases from Aspergillus oryzae, Trichoderma viride and Aspergillus niger. Multicell 185 has a cellulase activity, at pH 6.5, of 185,000 units/g. Assay Method: C₁-ase as well as CMC-ase. Multicel also has some xylanase activity.

EX 28000 enzymes product is a water dispersible blend of the extracts of Bacillus subtilis and Aspergillus oryzae. The product includes high concentrations of alpha-amylase, beta-glucanase (gumase), and hemi-cellulase. The product has an Amylolytic Activity of 28,000 BAU/gram, a Betaglucanase Activity of 12,000 Betaglucanase units/gram and a Hemicellulase Activity of 900 Hemicellulase units/gram, Although a primary enzyme associated with Bacillus subtilis exact is amylase, other useful hydrolases are often included in this product. These other enzymes catalyze the breakdown of complex carbohydrates other than starch. Hemicellulase activity attacks plant wall components. Beta-glucanase helps break down beta-linked glucose polymers often associated with grains, such as barley, oats, and wheat, and other products, including soy bean meal and locust bean gum. This additional digestive action is broadly classified as gumase activity. The presence of soluble calcium has a stabilizing effect on most enzymes of this type.

Based upon the above ingredients the feed additive contained 1.6×10¹⁴ colony forming units per 2000 (907.2 Kg) pounds of feed additive and an enzyme activity of 4.09×10⁹ per 2000 pounds (907.2 Kg) giving a ratio of enzyme activity to colony forming units of 1 to 1.16×10⁵.

Other ingredients in the feed additive of the present invention include 987.2 pounds (447.8 kg) of calcium carbonate, 400 pounds (181.4 kg) of corn gluten, 500 pounds (226.8 kg) of dried molasses and 24 pounds (10.9 kg) of mineral oil. The total weight of the feed additive was 2,000 pounds (907.2 Kg). Each ounce (28.35 g) of the feed additive contained 3×10⁹ colony forming units and 1.25×10⁵ units of enzyme activity. The numbers are approximate because of the instability of the colony forming units, and the presence of enzyme activity in addition to the enumerated enzyme activity.

### EXAMPLE 1

A total mixed ration (TMR) balanced for 70 pounds (31.6 kg) per day of milk product on was prepared as shown in TABLE 1. Based upon an assumed intake of 50 pounds (22.7 kg) a day, the Protein Edge (PE) feed additive of the present invention was added to four batches of the TMR at levels of 0.75, 1.0 and 1.5 ounces (0, 21.3, 28.4, 42.5 gms) per 50 pounds (22.7 kg) of TMR.

TABLE 1
Composition and Analysis of
The Total Mixed Ration

| Ingredient | % of Dry Matter (DM) |
|---|---|
| Mixed haylage | 24.0 |
| Corn silage | 42.0 |
| Advantage Silver (chicken feathers) | 2.0 |
| Soy Bean Meal, 48% | 8.4 |
| Barley, ground | 4.2 |
| Corn, ground | 9.2 |
| Wheat midds | 8.9 |
| Dicalcium Phosphate | .3 |
| Limestone | .6 |
| Salt | .3 |
| MgO | .1 |

Analyses

| | |
|---|---|
| Crude Protein (C Protein) | 14.6 |
| Neutral detergent fiber (NDF) | 35.1 |
| Acid detergent fiber (ADF) | 20.9 |
| Ether extract | 3.1 |
| Non-structural carbohydrate (NSC) | 33.2 |
| Ash | 5.9 |

Each of the four batches of TMR was fermented in quadruplicate using rumen conditions similar to those of a lactating dairy cow.

The effects of treatment levels are shown in Table 2

TABLE 2
Treatment Level oz (gm)

| | | | | |
|---|---|---|---|---|
| Nutrient | 0 (0) | .75 (21.3) | 1.0 (28.4) | 1.5 (42.6) |

Digestion Coefficients

| | | | | |
|---|---|---|---|---|
| Organic matter | 61.1 | 59.0 | 62.2 | 58.2 |
| Crude Protein (C. Protein) | 84.0 | 91.1 | 88.3 | 88.6 |
| NDF | 48.5 | 48.0 | 48.2 | 49.3 |
| ADF | 48.5 | 46.1 | 44.9 | 47.6 |
| Starch and Sugar | 81.7 | 80.4 | 84.7 | 84.7 |

Digestion of starch and sugar, along with protein, appears to increase with added levels of the additive of the present invention.

Protein utilization and microbial protein production are set forth in Table 3.

TABLE 3
Treatment Level oz (gm).

| | | | | |
|---|---|---|---|---|
| Item | 0 | .75 (21.3) | 1.0 (28.4) | 1.5 (42.6) |

Protein Utilization, Microbial Production and Efficiency

| | | | | |
|---|---|---|---|---|
| Crude Protein intake g/d | 14.6 | 14.6 | 14.7 | 15.1 |
| Ammonia, mg/d | 9.9 | 9.3 | 9.4 | 8.3 |
| By-Pass protein, g/d | 2.3 | 1.3 | 1.7 | 1.7 |
| Microbial protein g/d | 10.1 | 11.3 | 11.0 | 11.6 |
| Microbial + bypass protein | 12.5 | 12.6 | 12.7 | 13.3 |
| Lbs. microbial protein per | .176 | .202 | .189 | .213 |
| lb. of organic matter | | | | |
| digested | | | | |
| Lbs. microbial protein per | .229 | .258 | .245 | .258 |
| lb. starch + sugar + fiber | | | | |
| digested | | | | |
| Percent of digested feed | 82.3 | 84.7 | 84.2 | 86.5 |
| protein converted to | | | | |
| microbial protein | | | | |

These data show that the additive of the present invention stimulates microbial growth. More importantly, the additive is functioning by increasing the efficiency of microbial growth, that is the pounds of microbial protein produced per pound of organic matter or carbohydrate fermented in the rumen. Not only are these responses highly significant statistically, but they are linear. That means that the more additive that you feed, the greater the response and up to 1.5 oz (42.6 gm) the response shows no sign of tapering off.

The addition of 0.75 oz (21.3 gm) of the additive of the present invention results in 0.73 pounds (0.33 kg) of additional microbial protein per day. This protein is equivalent to or better than the best by-pass protein available. The additive of the present invention is a very good replacement, both in cost and safety, for fish meal, blood meal, bone meal or any other animal protein supplement.

### EXAMPLE 2

### Materials and Methods

### Cows and Diet

Forty multiparous Holstein cows between 70 and 180 days in milk were randomly assigned to one of two treatment groups: 1) Control ration balanced for bypass and total protein using traditional ingredients; and 2) Treatment ration balanced for bypass and total protein using the Protein Edge feed additive of the present invention at a rate of 2.0 oz (56.7 gm)/cow/day. The recommended rate was 1.0 oz (28.4 gm)/cow/day however based on analyses after the trial was commenced, it was decided the efficacy of the product was lower than anticipated and Protein Edge was increased to 2.0 oz (56.7 gm)/cow/day. The cows were housed in a free-stall for the duration of the experiment The experiment followed a crossover design with two 28-day periods, the final 7 days of each period were used to collect milk samples and other animal performance data.

It was the objective of the investigators to balance rations using similar ingredients for both control and treatment rations with only the elimination of bypass protein supplements, and the improvement of the starch and rumen degradable proteins for the enhanced microbial efficiency. Rations were balanced using Cornell, Penn, Miner (CPM) Dairy model (the commercial version of the Cornell Net Carbohydrate/Protein system) to match microbial yield for both treatment diets with and without EP5. Unfortunately, a miscommunication between the feed mill and investigators resulted in different ingredients used in the concentrate for the control and treatment diets (Table 4), The chemical composition of the Control and Treatment (Protein Edge) concentrates are presented in Table 5. Fortunately, the rations had similar nutrient profiles, microbial yields and efficiencies and approached the original objectives of the study. (Table 6). The increase in the microbial efficiency was accounted for by increasing the microbial maximum growth by 20% in the Cornell, Penn Miner Dairy model. Forages and feeds for the study were analyzed for routine analyses plus sugars & starches, CPM Dairy analyses and fermentation acids in the silages. The resultant rations both had 11 to 12% rumen degradable protein and the treatment rations resulted in less total protein and about 1 lb (0.45 kg) less bypass protein per day.

TABLE 4
Diet Composition (% DM)

| Ingredients | Control Diet | PE Diet |
|---|---|---|
| Corn silage | 30.55 | 30.51 |
| Hay crop silage | 15.28 | 15.26 |
| Cottonseed Wlint | 4.34 | 4.33 |
| Corn meal | 24.97 | 28.22 |
| Canola meal | 7.81 | 7.80 |
| Corn gluten meal | 2.47 | — |
| Cookie meal (Baking Industry Waste) | 1.83 | — |
| Soybean Whlroast | 2.41 | 1.63 |
| WheatGm DryHard Med | 1.84 | — |
| Soybean meal48CP | 1.85 | 3.92 |
| SQ-810 Bulk (sesqui carbonate buffer) | 1.05 | 1.32 |
| CornDistGrnLight | 0.95 | 1.19 |
| Limestone ground | 0.84 | 1.96 |
| Salt-bulk A | 0.41 | 0.52 |
| FCI 78 (dried animal and fish meal and poultry | 0.38 | — |
| feathers) | | |
| Protein Edge | — | 0.27 |
| Dical bulk | 0.35 | 0.43 |
| Megalac (calcium salts of palm oil distillate) | 1.12 | 1.00 |
| Urea | 0.35 | 0.39 |
| Calcium Sulfate | 0.21 | 0.01 |
| Zinpro 40 (trace minerals) | 0.04 | 0.05 |
| Magox (Magnesium oxide) | 0.05 | 0.03 |
| Tallow | 0.47 | 0.53 |
| MinVit (minerals and vitamins) | 0.45 | 0.63 |
| Total | 100.0 | 100.0 |

TABLE 5
Chemical Composition of Protein Concentrates
on a Dry Matter (DM) Basis

| | Control | PE |
|---|---|---|
| Chemical | Concentrate | Concentrate |
| DM (%) | 89.6 | 89.4 |
| Ash | 27.0 | 29.5 |
| Crude Protein (CP) | 37.5 | 37.8 |
| ADF | 10.3 | 9.4 |
| NDF | 15.8 | 14.9 |
| Non Fiber Carbohydrate (NFC) | 13.7 | 11.0 |
| Fat | 9.1 | 9.0 |
| Calcium | 4.03 | 5.81 |
| Phosphorus | 0.79 | 0.96 |
| Magnesium | 0.40 | 0.57 |
| Chloride | 1.72 | 2.23 |
| Potassium | 0.92 | 1.48 |

### Sampling and Analysis

### Feed

Throughout the experiment, cows were fed once daily (1300 h) at 110% of expected intake. Ration components were sampled weekly through the course of the experiment. The TMR and what the cattle did not eat (orts) for both groups were sampled weekly and analyzed as a composite for each period. A sub-sample of both TMR and orts were taken for dry matter determination and the remainder of the composite was sent for wet chemistry analysis. Amounts of feed offered and an estimate of refused feed was recorded daily during the collection period.

### Milk

The cows were milked three times per day in the milking parlor at 0500, 1300, and 2100. For each cow, milk yield was measured daily and a weekly average calculated for the last week of each period. Milk was sampled from the same milking on two consecutive days of each collection period and analyzed for total protein, fat composition, lactose, milk urea nitrogen and somatic cells.

### Body Condition Score

Body condition score was measured using the five-point scale where 1=thin to 5=fat and determined by two trained investigators on once day prior to the start of the first period and on the last day of each period

### Statistical Analysis

The effects of EP5 in the diet on milk and milk component production were analyzed using the fit model procedure of JMP® using the following model:
Yᵢⱼₖₗ=μ+Sᵢ+Cⱼ(Sᵢ)+Pₖ+Tₗ+eᵢⱼₖₗ

Where

μ=overall mean,

TABLE 6
Nutrient Profile of Study Rations

| Parameter | Control Diet | PE Diet |
|---|---|---|
| CPDM (%) (crude protein % dry matter) | 17.8 | 16.7 |
| Ruminal Undegradability (UIPIP) | 38.1 | 33.3 |
| Fat (%) | 6.0 | 5.8 |
| NDF (%) | 31.3 | 32.1 |
| eNDF (%)(neutral detergent fiber | 21.8 | 22.4 |
| effective) | | |
| NSC (%) | 39.7 | 38.7 |
| Rumen Balance | | |
| Peptide (% of requirement) | 121 | 98 |
| NH₃ (% of requirement) | 175 | 136 |
| Amino Acid Balance | | |
| Met (g) | −3.7 | −2.6 |
| Met (% mp) | 2.01 | 2.07 |
| Lys (g) | −38.4 | −24.3 |
| Lys (% mp) | 6.2 | 6.81 |
| Bacterial Yield CP (g/d) | 2278.31 | 2676.34 |
| Bacterial Growth Efficiency | | |
| g Bact N/kg Ferment CHO | 35.11 | 41.37 |

Sᵢ=effect of sequence (i=1 or 2),

Cⱼ(Sᵢ)=effect of cows nested in sequence (i=1 to 25),

Pₖ=effect of period (k=1 to 2),

Tₗ=effect of treatment (l=1 to 2), and

eᵢⱼₖₗ=residual, assumed to be normally distributed.

Dry mater intake was characterized but no statistical analysis was conducted since the cows were group fed.

### Results & Discussion

The results of the study are presented in Table 7. Estimated dry matter intake for animals fed Control and Protein Edge diets were 51.23 lbs (23.2 kg) and 53.36 lbs (24.2 kg)/cow/day, respectively. There were no differences in animal performance based on the diet the animals consumed. Cows held the same level of productivity on the concentrate containing Protein Edge as when they were fed the concentrate with bypass protein. Admittedly, other base ingredients of the concentrates differed and inferences about animal performance based on the diets fed cannot assume that animals will perform equally well with Protein Edge replacing animal protein in the concentrate. However, based on the nutrient parameters shown by the CPM nutritional model, the findings of this study are encouraging and support the concept of feeding Protein Edge to increase microbial protein yield and efficiency in lactating dairy cows. According to the CPM model, the Control and Protein Edge diets supplied sufficient metabolizable energy (ME) to support 96.5 (43.8 kg) and 91.8 lbs (41.6 kg) of milk respectively when milk composition was held constant at 3.5% milk fat and 3.3% milk protein. In addition, the CPM model found the Control and Protein Edge diets supplied sufficient metabolizable protein (MP) to support 77.9 (35.3 kg) and 76.8 lbs (34.8 kg) of milk respectively when milk composition was held constant. According to the results shown in Table 7, animals yielded higher levels of milk than the model predicted, however milk protein was lower for both diets.

The theoretical efficiency of producing milk is maximum when both efficiencies in rumen and tissue utilization are maximum. The new concepts require that minimal undegradability (RUP) must be specified in addition to total tissue protein requirement.

Lactation performance, milk composition and body condition score of Holstein cows fed an animal-protein based concentrate (control) or a non-animal based concentrate containing Protein Edge.

TABLE 7

| | |
|---|---|
| Control | PE |

| Parameter | N | LSmean | SE | LSmean | SE | p-value |
|---|---|---|---|---|---|---|
| Milk Yield (lbs/day) | 40 | 85.30 | 1.24 | 85.25 | 1.24 | 0.9751 |
| Milk Fat % | 39 | 3.80 | 0.08 | 3.86 | 0.07 | 0.5563 |
| Milk Protein % | 39 | 3.03 | 0.01 | 3.01 | 0.01 | 0.2952 |
| Milk Lactose % | 39 | 4.80 | 0.02 | 4.83 | 0.02 | 0.2606 |
| Milk Urea Nitrogen | 39 | 11.79 | 0.46 | 11.15 | 0.45 | 0.3229 |
| (MUN) mg/dl | | | | | | |
| Somatic Cell Count | 39 | 329.44 | 54.60 | 317.95 | 51.80 | 0.8795 |
| (SCC) (×1000) | | | | | | |
| BCS | 40 | 3.28 | 0.03 | 3.25 | 0.03 | 0.4289 |

### EXAMPLE 3

### Effects of the Addition of Protein Edge™ Protein Replacement on Microbial Metabolism in Continuous Culture of Rumen Contents

### Objectives

Continuous culture evaluations conducted at West Virginia University (Example 1) with LBJ Pakke's Protein Edge™ protein replacement showed that supplementation at a rate equivalent to 1 oz/head/day resulted in significant increases in microbial efficiency. The current study was conducted to evaluate the responses of the Protein Edge™ protein replacement run under similar dietary and experimental conditions.

### Procedures

The basal diet was prepared at West Virginia University to be as close to the 1996 diet as possible. Protein Edge was provided by LBJ Pakke and consisted of two products, which are identified as Protein Edge 1 (PE1) (2.25×10⁹ CFU/oz) and Protein Edge 2 (PE2) (9.60×10⁸ CFU/oz). Addition of both products was at the equivalent of 1 oz/head/day. Composition and analyses of the diets are shown in Tables 1 and 2, respectively. Protein Edge was used in both products with the only difference being colony forming units levels.

TABLE 8
Basal Diet Composition

| Ingredient | % of DM |
|---|---|
| Corn Silage | 42.02 |
| Mixed Haylage | 24.01 |
| Ground Corn | 9.21 |
| Wheat Midds | 8.89 |
| Soybean Meal, 48% | 8.39 |
| Rolled Barley | 4.20 |
| Protein Blend-WP2 | 2.00 |
| Dicalcium Phosphate | 0.29 |
| TM Salt | 0.29 |
| Magnesium Oxide | 0.10 |
| Limestone | 0.59 |

TABLE 9
Diet Analysis, % Dry Matter Basis.

| | Diet | | |
|---|---|---|---|
| Component | Control | Protein Edge 1 | Protein Edge 2 |
| Crude Protein | 14.0 | 14.3 | 14.6 |
| Soluble Protein, % CP | 23.7 | 23.1 | 22.5 |
| Neutral Detergent Fiber | 39.7 | 40.9 | 40.3 |
| Acid Detergent Fiber | 22.9 | 24.2 | 23.6 |
| Nonstructural Carbohydrate | 33.4 | 33.7 | 34.2 |
| Starch | 30.2 | 30.5 | 31.0 |
| Sugar | 3.2 | 3.2 | 3.2 |
| Ether Extract | 2.4 | 2.6 | 2.3 |
| Ash | 6.5 | 6.5 | 6.6 |
| Calculated Non-Fiber | 37.4 | 35.5 | 36.2 |
| Carbohydrate | | | |

Continuous culture fermentations were conducted using conditions simulating rumen parameters of lactating cows and the same as in the Protein Edge earlier evaluation. These conditions were:

| | |
|---|---|
| Liquid dilution rate | 12%/hr |
| Solids dilution rate | 3.57%/hr |
| Solids Retention Time | 28 hr |
| Feed Intake | 100 g DM/d |
| Feeding Frequency | 50 g DM, 2 times daily |
| Fermentation temperature | 39° C. |
| pH | Recorded at 2 hr intervals |

Each diet was fermented in triplicate 9-day fermentations, with effluent samples composited for analysis during the last three days.

Statistical contrasts included Control vs Treatments and Protein Edge 1 vs. Protein Edge 2 using GLM procedures of SAS.

### Results

Digestion coefficients of the dietary nutrients are shown in Table 10. Both treatments significantly enhanced digestion of ADF, with PE2 resulting in greater ADF digestion than PE1. Digestion of NSC was significantly greater on the PE2 diet compared to PE1. The differences in NDF and NSC digestion between PE1 and PE2 were not large so that the total carbohydrate digested was not different between the two treatments. The total carbohydrate digestion for both treatments was marginally greater (P=0.12) than for the control, as was crude protein digestion (P=0.11).

TABLE 10
Effects of the Treatments on Nutrient Digestion Coefficients.

| | Diet | | | P Values | |
|---|---|---|---|---|---|
| Item | Control | PE1 | PE2 | Control vs Trt | PE1 vs PE2 |
| Digestion, % | | | | | |
| Dry Matter | 63.0 | 67.1 | 61.2 | .58 | .04 |
| Organic Matter | 43.9 | 43.9 | 43.5 | .90 | .85 |
| Acid Detergent | 37.9 | 41.5 | 47.9 | .02 | .04 |
| Fiber | | | | | |
| Neutral Detergent | 31.5 | 35.6 | 34.2 | .18 | .59 |
| Fiber | | | | | |
| NSC¹ | 75.8 | 73.5 | 78.8 | .87 | .08 |
| CHO², g/day | 37.8 | 39.4 | 40.7 | .12 | .38 |
| Crude Protein | 66.0 | 76.7 | 69.9 | .11 | .18 |

¹Nonstructural carbohydrate (sugars + starch)
²Total carbohydrate digested (NDF + NSC), g/day

Total VFA production (Table 11) did not differ among rations. Butyric and valeric acid production was significantly decreased by both treatments, while isobutyrate was increased. The average fermentation pH is shown in Table 11. No differences due to treatments were found, but pH on all diets was below 6.0 for about 4 hours of the 12-hour post-feeding cycle.

TABLE 11
Effects of Treatments on Fermentation pH, Volatile Fatty Acid (VFA)
Production and Acetate-Propionate Ratio.

| | Diet | | | P Values | |
|---|---|---|---|---|---|
| Item | Control | PE1 | PE2 | Control vs Trt | PE1 vs PE2 |
| Average pH | 6.17 | 6.12 | 6.14 | .38 | .61 |
| VFA | | | | | |
| mmoles/day | | | | | |
| Total | 386 | 388 | 386 | .87 | .77 |
| Acetic | 213 | 219 | 214 | .39 | .34 |
| Propionic | 111 | 120 | 122 | .20 | .80 |
| Butyric | 48 | 38 | 38 | .08 | .99 |
| Isobutyric | 2.5 | 2.8 | 2.8 | .06 | .91 |
| Valeric | 9.6 | 6.9 | 7.9 | .01 | .23 |
| Isovaleric | 1.8 | 2.1 | 1.9 | .28 | .31 |
| A-P Ratio | 1.93 | 1.83 | 1.76 | .16 | .50 |

Microbial growth and nitrogen partitioning are show in Table 12. Total non-ammonia-nitrogen flow was enhanced by both treatments (P<0.01) compared to the control. This measurement contains both the by-pass feed protein plus the microbial protein, and indicates a considerably larger amount of total protein leaving the rumen due to the treatments. Since there was no difference in by-pass feed protein (NANMN) due to treatments, the increase in nitrogen flow was due solely to a significant increase in microbial growth caused by both treatments. The microbial N on PE1 was numerically greater than on PE2.

TABLE 12
Nitrogen Partitioning

| | | | | P Values | |
|---|---|---|---|---|---|
| | Diet | | | | PE1 |
| Item | Control | PE1 | PE2 | Control vs Trt | vs PE2 |
| Ammonia N, | 2.52 | 1.96 | 1.55 | .21 | .53 |
| mg/dl | | | | | |
| Non-ammonia | 2.47 | 2.54 | 2.59 | .0005 | .03 |
| N, g/d | | | | | |
| Microbial N, g/d | 1.60 | 1.93 | 1.79 | .04 | .28 |
| NANMN¹, g/d | 0.87 | 0.61 | 0.79 | .15 | .16 |

¹Non-Ammonia, Non-Microbial N (bypass feed N)

Efficiencies of nutrient use for microbial growth are shown in Table 13. Both treatments significantly enhanced microbial efficiency in terms of digested DM and OM. While carbohydrate efficiencies did not achieve statistical significance, the g microbial N/kg carbohydrate digested was numerically greater for both treatments as well. The conversion of digested feed N to microbial N was enhanced significantly by both treatments compared to the control.

Partial analysis of the composition of the microbes (Table 13) shows no significant differences due to the treatments.

TABLE 13
Grams of Microbial N produced per kg Digested DM, OM and
Carbohydrate, and Efficiency of N Uptake.

| | Diet | | | P Values | |
|---|---|---|---|---|---|
| Item | Control | PE1 | PE2 | Control vs Trt | PE1 vs PE2 |
| G Mic. N/Kg: | | | | | |
| Digested DM | 25.4 | 28.7 | 29.3 | .02 | .68 |
| Digested OM | 38.9 | 47.0 | 44.3 | .03 | .36 |
| Digested CHO¹ | 42.3 | 49.4 | 44.1 | .30 | .28 |
| CP Efficiency² | 94.9 | 96.8 | 96.9 | .07 | .93 |

¹NDF + NSC digested
²% degraded feed N as Microbial N

TABLE 14
Analysis of Microbes

| | Diet | | | P Values | |
|---|---|---|---|---|---|
| Item | Control | PE1 | PE2 | Control vs Trt | PE1 vs PE2 |
| Nitrogen, % of | 7.89 | 7.84 | 8.11 | .74 | .35 |
| DM | | | | | |
| Ash, % of DM | 14.01 | 15.73 | 15.48 | .31 | .89 |
| RNA-N, % of | 7.76 | 7.85 | 7.88 | .84 | .97 |
| Mic.N | | | | | |

### Implications and Conclusions

Protein Edge had a number of effects on microbial growth and metabolism. Both PE1 and PE2 increased digestions of ADF and tended to increase digestion of NDF. Although comparison of the products with each other revealed PE2 to have an advantage over PE1 in terms of increased digestion of ADF and NSC, the comparative results were often inconsistent. For example, digestion of DM was significantly greater for PE1 than PE2 with protein digestion following a similar trend. The advantage of PE1 over PE2 also was observed in total NAN flow/day. These data led us to conclusions that (1) both products had significant positive effects on nutrient digestion, microbial growth and efficiency, and (2) there was insufficient conclusive evidence to suggest one product was superior to the other.

In comparing this study with the previous trials done with Protein Edge, the current Protein Edge products appear to have an advantage in increasing digestibility of ADF, and tending to increase NDF and protein, while the PE products had no effect on digestion coefficients. PE, PE1 and PE2 positively affected microbial growth and efficiency. In making these comparisons, we will use values scaled up to those of a cow consuming 25 kg DM/day, and will focus on the average responses to PE, PE1 and PE2. Comparisons of microbial efficiency based on organic matter were:

| Protein Edge 1 & 2 | | | | | |
|---|---|---|---|---|---|
| | | Percent | Protein Edge | | |
| Treatment | Efficiency | Change | Treatment | Efficiency | Percent Change |
| Control | 38.9 | — | Control | 28.13 | — |
| PE1 | 47.0 | +20.8 | Level 1 | 32.28 | +14.8 |
| PE2 | 44.3 | +13.9 | Level 2 | 30.21 | +7.4 |
| | | | Level 3 | 34.04 | +21.0 |
| PE mean | 45.7 | +17.4 | PE mean | 32.18 | +14.38 |

PE, PE1 and PE2 gave very similar average percent increases in microbial efficiency based on organic matter digestion. These calculations also could be done using DMD or carbohydrate digestion. In the current study, the presence of an outlier NDF digestion in one repetition resulted in no significant responses for CHO efficiency. We did the same calculations as for OM efficiency for comparative purposes, and found the average percent increases in efficiency per unit of carbohydrate digested to be 10.5 and 10.8 for PE (1,2) and PE, respectively.

From the previous study it can be calculated that the addition of 0.75 oz of PE to the ration of a cow consuming 25 kg DM/day would result in 363 g additional microbial protein/day. Based on OM digestion, the calculations for PE in the current study were:

| | OM intake, | OM | | | |
|---|---|---|---|---|---|
| Treatment | kg | Digestion | OM Eff | g Mic Prot/d | g increase |
| Control | 23.38 | 43.9 | 38.9 | 2494 | — |
| PE1 | 23.38 | 43.9 | 47.0 | 3013 | 519 |
| PE2 | 23.38 | 43.5 | 44.3 | 2838 | 344 |

The average increase in microbial protein was 432 g, which is a bit higher than found for the previous study. Keep in mind that this is microbial protein of a quality difficult to replace with feed protein. Assuming, as in the previous study, fishmeal at $450/ton and 70% by-pass were used, about 1.4 lbs/cow per day would be required, with a value of 31.5 cents.

One objective was to determine if PE could be used in a diet of low protein and achieve microbial responses normally seen on a higher protein diet. Taking into account the average microbial protein increase over the control (432 g), the percent increase in feed protein needed in the control diet (at 33% by-pass) would be 5% i.e., 1190/total CP.

The results of this study show PE (1,2) to be somewhat superior to PE in promoting microbial growth, and have effects on nutrient digestion not seen with PE.

What appears to be happening is that the enzymes digest fiber very rapidly for the added bacteria, and the naturally occurring bacteria in the rumen, multiply and become significant amounts of microbial protein.

APPENDIX
Forage Analysis - % Dry Matter Basis

| Component | Corn Silage | Haylage |
|---|---|---|
| Dry Matter | 38.96 | 48.64 |
| Crude Protein | 5.76 | 12.79 |
| Soluble Protein, % CP | 36.71 | 37.28 |
| ADFIP, % CP | 6.56 | 16.64 |
| NDFIP, % CP | 7.23 | 37.78 |
| NDF (no sodium sulfite) | 44.88 | 57.52 |
| Lignin, % NDF | 5.64 | 11.61 |
| ADF | 25.87 | 38.22 |
| NSC* | 34.99 | 10.79 |
| Sugar | 1.76 | 5.10 |
| Starch | 33.23 | 5.69 |
| Ash | 3.68 | 10.05 |
| Fat | 2.84 | 2.46 |
| NFC** | 42.84 | 17.18 |
| Ca, % | .25 | 0.77 |
| P, % | .21 | 0.39 |
| Mg, % | .11 | 0.33 |
| K, % | 1.17 | 3.19 |
| Volatile Fatty Acids: % DM | | |
| Acetic | 2.22 | 1.09 |
| Propionic | .37 | .02 |
| Isobutyric | .07 | .04 |
| Butyric | .02 | .007 |
| Lactic | 3.28 | 3.30 |
| Total VFAs | 5.97 | 4.47 |

In the following claims, the upper limits of both enzyme and bacteria are determined by the law of diminishing return and can be easily determined by testing. Excess enzyme is known not to be beneficial. Excess bacteria are not economically feasible. Because the bacteria multiply so fast in the rumen in the presence of the enzyme, bacteria levels are not as important as enzyme levels. Only the animal protein causing disease needs to be replaced in the diet of the cattle.

### INDUSTRIAL APPLICABILITY

The present invention is a cattle feed additive derived from non animal sources which can be used to replace animal feed additives derived from animal sources. The additive of the present invention results in a savings compared to an animal protein of comparable or inferior quality. The additive of the present invention results in a better amino acid balance in the digestive tract of cattle resulting in more meat and milk produced and less pollution in the manure. Excess quantities over the normal balance of amino acids are passed as pollution in the cattle manure. The diseases, such as “mad cow”, carried by some animal protein additives, are not present in the additives of the present invention.

1. A method for enhancing the production of microbial protein in the rumens of cattle characterized by addition of a feed additive to cattle feed, the additive made by combining fibrolytic enzymes having enzyme activity and one or more species of lactobacillus bacteria having colony forming units wherein the ratio of enzyme activity to colony forming units has a value, of at least about 1 unit of digestive enzyme activity to every 10⁵ colony forming units, based upon the ingredients prior to combination, the combination not being ensiled after being combined, and feeding the additive combined with the cattle feed to the cattle.

2. The method of claim 1 further characterized by the ratio of enzyme activity to colony forming units having a value of at least 2 units of enzyme activity to every 10⁶ colony forming units.

3. The method of claim 1 further characterized by the lactobacillus bacteria being selected from the group comprising Lactobacillus Acidophilus, Lactobacillus Plantarum, and Lactobacillus Brevis, and mixtures thereof.

4. The method of claim 1 further characterized by the fibrolytic enzymes being selected from the group comprising cellulases, xylanase, hemi-cellulase and mixtures thereof.

5. A method of making and using cattle feed characterized by replacing previously used bypass protein in the cattle feed with an additive, the additive containing a sufficient amount of a mixture of one or more specie of lactobacillus bacteria and one or more types of fibrolytic enzymes, to produce, when fed to cattle, at least enough microbial protein in each head of cattle to be at least equivalent to one half pound (0.23 kg) of animal protein fed to each of the cattle per day, the additive not being ensiled after the bacteria and enzymes are added and feeding the cattle feed containing the additive to a head of cattle to form at least a sufficient amount of microbial protein in the rumen of the head of cattle to be at least equivalent to one half pound (0.23 kg) of animal protein fed to the head of cattle per day, assuming that each of the cattle are mature and of an average weight for cattle.

6. The method of claim 5 further characterized by the lactobacillus bacteria being selected from the group consisting of Lactobacillus Acidophilus, Lactobacillus Plantarum, and Lactobacillus Brevis, and mixtures thereof, and the protein byproducts replaced are selected from the group consisting of nerve, brain, blood, bone and meat containing byproducts.

7. The method of claim 5 further characterized by the lactobacillus bacteria being a mixture of Lactobacillus Acidophilus, Lactobacillus Plantarum, and Lactobacillus Brevis.

8. The method of claim 5 further characterized by the one or more digesting enzymes being selected from the group consisting of xylanase, and cellulases derived from Trichoderma viride, Aspergillus oryzae, Aspergillus niger, and Bacillus subtilis.

9. The method of claim 5 further characterized by the one or more digesting enzymes being a mixture of xylanase, and cellulases derived from Trichoderma viride, Aspergillus oryzae, Aspergillus Niger, and Bacillus subtilis.

10. A method of enhancing the conversion of cattle feed to microbial protein in the rumen of a head of cattle characterized by incorporating an additive containing a sufficient amount of a mixture of one or more species of lactobacillus bacteria and one or more types of digesting enzymes into the cattle feed to form at least a sufficient amount of microbial protein in the rumen of the head of cattle to be at least equivalent to one half pound (114 kg 0.23 kg) of animal protein fed to each of the cattle per day and feeding the cattle feed containing the additive to a head of cattle to form at least a sufficient amount of microbial protein in the rumen of the head of cattle to be at least equivalent to one half pound (0.23 kg) of animal protein fed to the head of cattle per day.

11. The method of claim 10 further characterized by the lactobacillus bacteria being selected from the group consisting of Lactobacillus Acidophilus, Lactobacillus Plantarum, and Lactobacillus Brevis, and mixtures thereof and the amount of microbial protein formed to be at least equivalent to one half pound (0.23 kg) of animal protein fed to each of the cattle per day.

12. The method of claim 10 further characterized by the lactobacillus bacteria being a mixture of Lactobacillus Acidophilus, Lactobacillus Plantarum, and Lactobacillus Brevis.

13. The method of claim 10 further characterized by the one or more digesting enzymes being selected from the group consisting of xylanase, hemi-cellulase and cellulases derived from Trichoderma viride, Aspergillus oryzae, Aspergillus niger, and Bacillus subtilis.

14. The method of claim 10 further characterized by the one or more digesting enzymes being a mixture comprised of xylanase, and cellulases derived from Trichoderma viride, Aspergillus oryzae, Aspergillus Niger, and Bacillus subtilis.

15. A method for enhancing the production of microbial protein in the rumen of a head of cattle characterized by combining a sufficient amount of one or more strains of lactobacillus bacteria and one or more types of digesting enzymes having an enzyme activity of at least 10⁴ digestive units per oz (28.35 g) based upon the ingredients prior to combination into an additive, introducing the additive without ensiling into cattle feed, feeding the feed containing the additive without ensiling to the head of cattle, a daily ration of feed fed to each head of cattle containing a sufficient amount of one or more strains of lactobacillus bacteria and one or more types of digesting enzymes having an enzyme activity of at least 10⁴ digestive units per oz (28.35 g) to enhance the conversion of the cattle feed and the bacteria in the rumen to microbial protein.

16. The method of claim 15 further characterized by the enzymes being present at a level sufficient to produce an enzyme activity of from 10⁴ to 10⁸ units per gram of cattle feed and the lactobacillus bacteria being present at a level sufficient to increase the yield of microbial protein in the rumen.

17. The method of claim 15 further characterized by microbial protein being produced in the cattle by interaction of the bacteria and enzymes, the bacteria being added at a level of from 10⁶ to 10¹⁰ colony forming units per gram of cattle feed and enzymes being added at a level sufficient to produce a digestive enzyme activity of from 10⁶ to 10⁷ units per gram of cattle feed based upon the amount being added.

18. A method of reducing runoff of water soluble nitrogen compounds from cattle manure characterized by incorporating a sufficient amount of a mixture of one or more species of lactobacillus bacteria and one or more types of digesting enzymes into an additive and combining the additive with cattle feed, and feeding the combination to cattle to form at least a sufficient amount of microbial protein to be at least equivalent to one fourth pound (0.11 kg) of animal protein fed to each of the cattle per day, the combination not being ensiled after incorporation of a sufficient amount of the mixture.

19. A method of improving the amino acid balance in the rumen characterized by combining a sufficient amount of one or more strains of lactobacillus bacteria and one or more types of cellulose and/or hemi-cellulose digesting enzymes in an additive, adding the additive to cattle feed without ensiling, feeding the cattle feed containing the additive to cattle to convert the cellulose and/or hemi-cellulose and nitrogen in the cattle feed to microbial protein in the rumen of cattle, the microbial protein having an improved amino acid balance over the amino acid balance of the microbial protein normally occuring in the rumen, the cattle feed being free of a surfactant on a carrier, and not being ensiled after the combination of the bacteria and enzymes.

20. A method for enhancing the production of microbial protein in the rumen characterized by combining a feed additive containing a combination of fibrolytic enzymes having enzyme activity and one or more species of bacteria having colony forming units wherein the ratio of enzyme activity to colony forming units has a value, prior to combination of at least about 1 unit of digestive enzyme activity to every 10⁵ colony forming units, adding the additive to animal feed, and feeding the animal feed containing the additive to a ruminant, the additive not being ensiled after the combining the enzymes and the bacteria.

21. The method of claim 20 further characterized by the feed additive containing a cellulose and/or a hemi-cellulose digesting enzyme, and the additive not being ensiled prior to being feed to a ruminant.

22. A method of making and using cattle feed characterized by replacing previously used bypass protein in the cattle feed with an additive, the additive containing a sufficient amount of one or more types of fibrolytic enzymes, to produce, when fed to cattle, at least enough microbial protein in each head of cattle to be at least equivalent to one half pound (0.23 kg) of animal protein fed to each of the cattle per day, assuming that each of the cattle are mature and of an average weight for cattle and feeding the cattle feed containing the additive to cattle to produce at least enough microbial protein in each head of cattle to be at least equivalent to one half pound (0.23 kg) of animal protein per day and the additive not being ensiled prior to being feed to a ruminant.
